(19) United States
(12) Patent Application Publication
(10) Pub. No.: US 2023/0313354 A1
(43) Pub. Date: Oct. 5, 2023

(54) COBALT-CHROMIUM ALLOY MEMBER, METHOD OF PRODUCING THE SAME, AND MEDICAL OR AEROSPACE DEVICE

(71) Applicant: NATIONAL INSTITUTE FOR MATERIALS SCIENCE, Tsukuba-shi, Ibaraki (JP)

(72) Inventors: Koichi TSUCHIYA, Tsukuba-shi, Ibaraki (JP); Takahiro SAWAGUCHI, Tsukuba-shi, Ibaraki (JP)

(21) Appl. No.: 18/011,745

(22) PCT Filed: Jul. 13, 2021
(86) PCT No.: PCT/JP2021/026241

§ 371 Date: Dec. 20, 2022

(30) Foreign Application Priority Data
Jul. 17, 2020 (JP) 2020-122529

(51) Int. Cl. C22F 1/10; C22C 19/07; C21D 9/08; C21D 9/52

(52) U.S. Cl. CPC C22F 1/10; C22C 19/07; C21D 9/08; C21D 9/525

(57) ABSTRACT

[Object] To provide a cobalt-chromium alloy member suitable for use in a medical or aerospace device.

[Solving Means] There is provided a cobalt-chromium alloy member, which is obtained by performing heat treatment for 1 minute or more and 60 minutes or less at a temperature exceeding a recrystallization temperature of a cobalt-chromium alloy material and not more than 1100° C. on a cobalt-chromium alloy as-processed material obtained by causing a cobalt-chromium alloy material to be subjected to cold plastic working into a predetermined shape, the cobalt-chromium alloy material having a composition of, in terms of mass %, 23 to 32% of Ni, 37 to 48% of Co, and 8 to 12% of Mo, a remainder thereof containing Cr and an unavoidable impurity, the composition satisfying a relationship of 20≤[Cr %]+[Mo %]+[unavoidable impurity %]≤40, the cobalt-chromium alloy member having a tensile strength of 800 to 1200 MPa, a uniform elongation of 25 to 60%, and a breaking elongation of 30 to 80%.

### TECHNICAL FIELD

The present invention relates to a cobalt-chromium alloy member suitable for use in medical devices such as stents, medical tubes, and medical guide wires and industrial materials in the aerospace field. In particular, the present invention relates to improvement of a cobalt-chromium alloy material that has excellent corrosion resistance, excellent biocompatibility, high strength, and excellent ductility, and is suitable for indwelling medical devices.

### BACKGROUND ART

As a metal member used in medical devices, particularly, a metal member to be implanted in the body, a metal having excellent corrosion resistance, excellent biocompatibility, and high mechanical properties is desired, and stainless steel, a nickel-titanium alloy, a cobalt-chromium alloy, or the like has been used. As such a biocompatible alloy, for example, a dental casting cobalt-chromium alloy (JIS T6115) has been known. As a nickel-containing alloy, a dental stainless steel wire (JIS T6103) has been known.

Among cobalt-chromium alloy members, a stent is a hollow tubular object intended to dilate stenosed internal vessels and maintains the dilated vessels and is roughly classified into a self-expandable stent and a balloon-expandable stent.

The self-expandable stent is fixed to the tip of a catheter and given self-expandability by using a superelastic alloy/shape memory alloy from the catheter at a predetermined position. For example, a stent using a nickel-titanium alloy has been put to practical use.

The balloon-expandable stent is a stent that is fixed to a balloon catheter by reducing the tube diameter and increases the tube diameter by expanding the balloon at a predetermined position. Mainly, stainless steel SUS316L and a cobalt-chromium alloy have been put to practical use. For example, in the case where stenosis occurs in a blood vessel, the stent is placed after dilating the stenosed portion by the balloon catheter and is used to support the inner wall of the blood vessel from the inside and prevent restenosis. Regarding the insertion of the stent, the stent is attached to the tip of the catheter in a reduced-diameter state outside the deflated balloon and is inserted into the blood vessel together with the balloon portion. After the balloon portion is positioned at the stenosed site, the balloon portion is inflated to expand the stent, the stent is left while the stenosed portion is dilated, and the balloon catheter is withdrawn.

As an alloy for balloon-expandable stents, ASTMF90-14 (Co-20Cr-15W-10Ni alloy (L605 alloy), ASTMF562-13 (Co-20Cr-10Mo-35Ni alloy (MP35N alloy)), and SUS316L have been known as surgical implant materials.

Meanwhile, rupture of the implanted metal in the field of orthopedic surgery and premature rupture of a stent in the field of cardiovascular medicine have been reported and there is a demand for a metal member having more excellent fatigue properties. We have proposed an alloy having improved low cycle fatigue properties with respect to an L-605 (Co-20Cr-15W-10Ni) alloy and an MP35N (Co-20Cr-10Mo-35Ni) alloy that are most commonly used as coronary stent materials (see Patent Literature 1). This alloy has a composition of, in terms of mass %, 10 to 27% of Cr, 3 to 12% of Mo, and 22 to 34% of Ni and a remainder thereof substantially contains Co and an unavoidable impurity, and Co is desirably 37 to 48%.

A guide wire assists in inserting a diagnostic or therapeutic catheter used in a blood vessel to a predetermined position in the blood vessel and has a structure in which a thin wire is wound around a core wire. The guide wire is required to have torque transmissibility that the rotation of the tip follows the rotation of the hand and sufficient strength and ductility so that it does not rupture during treatment.

### CITATION LIST

### Patent Literature

Patent Literature 1: Japanese Patent Application Laid-open No. 2019-147982

### Non-Patent Literature

[Non-Patent Literature 1] Comparing and Optimizing Co—Cr Tubing for Stent Applications”, Medical Device Materials II, p. 274-278, (2004) ASM International.
[Non-Patent Literature 2] P. Zhang, S. X. Li, Z. F. Zhang, Materials Science and Engineering, A529(2011)62-73
[Non-Patent Literature 3] Fort Wayne Metals, Inc. (Fort Wayne, Indiana, USA) homepage, Materials, High Performance Alloys, L-605 https://www.fwmetals.jp/materials/high-performance-alloys/1-605/
[Non-Patent Literature 4] ASM Aerospace Specification Metals Inc. (Pompano Beach, Florida, USA) homepage, AISI Type 316 Stainless Steel, annealed sheet http://asm.matweb.com/search/SpecificMaterial.asp bassnum=MQ316A

### DISCLOSURE OF INVENTION

### Technical Problem

L605, which is a Co—Cr alloy currently used, and a Ti—Ni alloy are materials that are difficult to cold work and the processing cost is very high as compared with SUS316.

Further, recently, a cobalt-chromium alloy member that is suitable for a medical device and an aerospace device and has high mechanical strength and ductility is desired.

In particular, there is a demand for the use of indwelling medical devices such as stents in blood vessels having fine and complicated shapes such as nerve vessels and cerebral vessels. In order to achieve this, it is necessary to thin, using a thin tube, the strut that is a metal portion of the stent, and a material having as high strength as possible is required in order to achieve a sufficient blood-vessel-holding force. This also leads to a reduction in the amount of metal placed in the body.

Using as thin a wire as possible as a guide wire makes it easier to insert the guide wire into fine blood vessels, but it needs to have as high strength as possible in order to achieve further favorable torque transmissibility. Further, a material having ductility is desirable to prevent rupture during use.

It is an object of the present invention to provide a cobalt-chromium alloy member suitable for use in a medical device or aerospace device.

In particular, it is another object of the present invention to provide a cobalt-chromium alloy member suitable for a guide wire that makes it easier to insert an indwelling medical device such as a stent into fine blood vessels.

### Solution to Problem

In order to achieve the above-mentioned object, a cobalt-chromium alloy member according to the present invention has employed the following configuration.

[1] A cobalt-chromium alloy member, which is obtained by performing heat treatment for 1 minute or more and 60 minutes or less at a temperature exceeding a recrystallization temperature of a cobalt-chromium alloy material and not more than 1100° C. on a cobalt-chromium alloy as-processed material obtained by causing the cobalt-chromium alloy material to be subjected to cold plastic working into a predetermined shape, the cobalt-chromium alloy material having a composition of,
in terms of mass %, 23 to 32% of Ni, 37 to 48% of Co, and 8 to 12% of Mo, a remainder thereof containing Cr and an unavoidable impurity, the composition satisfying a relationship of

20≤[Cr %]+[Mo %]+[unavoidable impurity %]≤40,
the cobalt-chromium alloy member having a tensile strength of 800 to 1200 MPa, a uniform elongation of 25 to 60%, and a breaking elongation of 30 to 80%.

[2] The cobalt-chromium alloy member according to [1] may be obtained by performing heat treatment for 1 minute or more and 60 minutes or less at a temperature of 900° C. or more and 1100° C. or less on a cobalt-chromium alloy as-processed material obtained by causing the cobalt-chromium alloy material to be subjected to cold plastic working into a predetermined shape, the cobalt-chromium alloy material having a composition of,
in terms of mass %, 25 to 29% of Ni, 37 to 48% of Co, and 9 to 11% of Mo, a remainder thereof containing Cr and an unavoidable impurity, the composition satisfying a relationship of

23≤[Cr %]+[Mo %]+[unavoidable impurity %]≤38,
the cobalt-chromium alloy member having a tensile strength of 850 to 1200 MPa, a uniform elongation of 50 to 60%, and a breaking elongation of 60 to 80%.

[3] The unavoidable impurity according to [1] or [2] may contain, in terms of mass %, 1.0% or less of Ti, 1.0% or less of Mn, 1.0% or less of Fe, 1.0% or less of Nb, 1.0% or less of W, 0.5% or less of Al, 0.1% or less of Zr, 0.01% or less of B, and 0.1% or less of C as contents of Ti, Mn, Fe, Nb, W, Al, Zr, B, and C.

[4] The cobalt-chromium alloy member having the composition according to any one of [1] to [3] may have a crystal structure consisting of a face-centered cubic lattice (fcc) or a crystal structure consisting of a face-centered cubic lattice (fcc) and a hexagonal lattice (hcp), an average crystal grain size of 5 to 30 m, and a band-like deformed zone tissue.

[5] A medical device or an aerospace device, which uses the cobalt-chromium alloy member according to any one of [1] to [4].

[6] The medical device according to [5] may include a stent, a tube, a wire, or an implant.

[7] A method of producing a cobalt-chromium alloy member, characterized by including:
a cobalt-chromium alloy material having a composition of,
in terms of mass %, 23 to 32% of Ni, 37 to 48% of Co, and 8 to 12% of Mo, a remainder thereof containing Cr and an unavoidable impurity, the composition satisfying a relationship of

20≤[Cr %]+[Mo %]+[unavoidable impurity %]≤40;
homogenizing the prepared cobalt-chromium alloy material at 1100° C. to 1300° C.;
causing the homogenized cobalt-chromium alloy material to be subjected to cold plastic working into a tubular shape or a wire shape to obtain a cobalt-chromium alloy as-processed material; and
performing heat treatment for 1 minute or more and 60 minutes or less at a temperature exceeding a recrystallization temperature of the cobalt-chromium alloy material and not more than 1100° C. on the cobalt-chromium alloy as-processed material that has been subjected to cold plastic working.

### Advantageous Effects of Invention

The cobalt-chromium alloy member according to the present invention has excellent mechanical properties such as improved strength and ductility by heat treatment exceeding the recrystallization temperature after being subjected to cold plastic working and is more reliable than existing products. For this reason, for example, when an indwelling medical device such as a stent is prepared using the cobalt-chromium alloy member according to the present invention, the reliability of the stent during mounting is enhanced and the mounting of the stent on an affected area is made easier.

In the cobalt-chromium alloy member according to the present invention, the face-centered cubic lattice (fcc) phase is stabilized by causing an alloy containing Co, Ni, Cr, and Mo as main constituents to be subjected to cold plastic working and then performing heat treatment exceeding the recrystallization temperature thereon. As a result, in the formed fcc phase, when the cobalt-chromium alloy member deforms, an fcc twin deformation and a deformation-induced transformation from an fcc to a hexagonal lattice (hcp) occurs and high work hardenability and excellent mechanical strength/ductility are exhibited.

Note that in the case where the cobalt-chromium alloy member according to the present invention further contains solute atoms such as Mo and Nb, segregation at dislocation cores or stacking faults of extended dislocations is capable of making it difficult for cross slip to occur and work hardening further increases the mechanical strength.

### BRIEF DESCRIPTION OF DRAWINGS

FIG. 1 is a comparative diagram of a low cycle fatigue life of a cobalt-chromium alloy material used in the present invention.

FIG. 2 shows appearance photographs of a tube as a cobalt-chromium alloy as-processed material (top) according to an Example of the present invention and a cobalt-chromium alloy member (bottom) on which heat treatment has been performed for 5 minutes at 1050° C., Part (a) being a whole photograph, Part (b) being an enlarged photograph of the main portion.

FIG. 3 is a stress-strain diagram obtained by a tensile test of an as-processed material prepared by causing the cobalt-chromium alloy material according to the Example of the present invention to be subjected to cold working into a tubular shape, a heat-treated material that is a cobalt-chromium alloy member, and an L605 alloy tube that is a comparative material.

FIG. 4 is a diagram comparing yield stresses, tensile strengths, and elongations of the as-processed material prepared by causing the cobalt-chromium alloy material according to the Example of the present invention to be subjected to cold working into a tubular shape, the heat-treated material that is a cobalt-chromium alloy member, and the L605 alloy tube that is a comparative material with each other.

FIG. 5 is a crystal orientation analysis image of the cobalt-chromium alloy material used in the present invention, which is obtained by a scanning electron microscope.

FIG. 6 shows inverse pole maps obtained by an electron backscatter diffraction (EBSD) method of the as-processed material prepared by causing the cobalt-chromium alloy material to be subjected to cold working into a tubular shape (a) and the heat-treated material thereof (b).

FIG. 7 shows appearance photographs of a wire as the cobalt-chromium alloy as-processed material according to the Example of the present invention, Part (a) being a whole photograph, Part (b) being an enlarged photograph of the main portion.

FIG. 8 is a stress-strain diagram obtained by a tensile test of the wire as the cobalt-chromium alloy member according to the Example of the present invention.

FIG. 9 is an appearance photograph of a stent obtained by performing laser processing on the tube as the cobalt-chromium alloy member according to the Example of the present invention.

### MODE(S) FOR CARRYING OUT THE INVENTION

[Overview of Present Invention]

A cobalt-chromium alloy member according to the present invention is obtained by performing specific heat treatment exceeding a recrystallization temperature on a cobalt-chromium alloy as-processed material obtained by causing a cobalt-chromium alloy material having a specific composition to be subjected to cold plastic working (hereinafter, referred to simply also as “cold working”) into a predetermined shape.

As a result, a cobalt-chromium alloy member exhibiting high work hardenability and excellent mechanical strength/ductility is obtained.

Hereinafter, details of the present invention will be described.

[Details of Present Invention]

(Cobalt-Chromium Alloy Material)

A cobalt-chromium alloy material according to the present invention contains Ni, Co, Mo, Cr, and an unavoidable impurity.

The unavoidable impurity is not an intentionally added constituent but is a constituent that is unavoidably mixed due to the material or process. The constituents of the unavoidable impurity are not particularly limited and may be, for example, Ti, Mn, Fe, Nb, W, Al, Zr, C, and the like, but do not necessarily need to contain them.

Further, the cobalt-chromium alloy material according to the present invention is not particularly limited as long as it has a specific composition range, and may be homogenized, subjected to hot working such as hot rolling and hot forging, or processed into a specific shape by cutting or the like, as will be described below.

The reason for limiting the composition range of the cobalt-chromium alloy material according to the present invention will be described below.

Note that the content of each constituent of the cobalt-chromium alloy material is the content (mass %, referred to simply as “%” below.) when the entire cobalt-chromium alloy material is 100 mass %.

Further, the numerical range in the present invention includes the upper limit value and the lower limit value. The same applies not only to the composition range shown below but also to the range of temperature treatment, the range of a tensile strength, and the range of a breaking elongation and a uniform elongation.

Ni (nickel) has effects of stabilizing the face-centered cubic lattice phase, maintaining the workability, increasing the corrosion resistance, improving the low cycle fatigue life, and improving the strength and ductility by heat treatment exceeding the recrystallization temperature after cold working. However, since it is difficult to achieve the effect of improving the strength and ductility by heat treatment when the content of Ni is less than 23% and it is difficult to achieve the effect of improving the strength and ductility by heat treatment when the content of Ni exceeds 32% in the composition range of Co, Cr, and Mo in the cobalt-chromium alloy material according to the present invention, the content of Ni in the present invention is 23 to 32%, favorably 25 to 29%. As a result, the effect of further improving the strength and ductility can be achieved.

Co (cobalt) itself has large work hardenability and has the effects of reducing the notch brittleness, increasing the fatigue strength, increasing the high-temperature strength, improving the low cycle fatigue life, and improving the strength and ductility by heat treatment exceeding the recrystallization temperature after cold working.

When the content of Co is less than 37%, the effect of Co is weak. In the present composition, when the content of Co exceeds 48%, the matrix becomes too hard and difficult to process and the effect of improving the strength and ductility by heat treatment exceeding the recrystallization temperature after cold working is lost. For this reason, the content of Co in the present invention is 37 to 48%, favorably 40 to 45%. As a result, the effect of further improving the strength and ductility is achieved.

Mo (molybdenum) has the effects of being dissolved in the matrix to strengthen this, increasing the work hardenability, and increasing the corrosion resistance in coexistence with Cr. However, a desired effect cannot be achieved when the content of Mo is less than 8%, and the workability is rapidly reduced and a brittle a phase is likely to be generated when the content of Mo exceeds 12%. For this reason, the content of Mo in the present invention is 8 to 12%, favorably 9 to 11%. As a result, the effect of further improving the strength and ductility is achieved.

When the total content of Cr, Mo, and the unavoidable impurity is less than 20% when the entire cobalt-chromium alloy material is 100%, the hexagonal lattice (hcp) phase becomes table. When the total content exceeds 40%, the face-centered cubic lattice (fcc) phase becomes unstable and a body-centered cubic lattice (bcc) phase tend to appear. That is, in the case where the total content of Cr, Mo, and the unavoidable impurity is not 20 to 40%, the fcc phase is difficult to stabilize, the fcc twin deformation and the deformation-induced transformation from fcc to hcp are difficult to occur when the cobalt-chromium alloy member thus obtained is deformed, and excellent ductility and a low cycle fatigue life cannot be achieved. For this reason, the total content of Cr, Mo, and the unavoidable impurity in the present invention is 20 to 40%, favorably 23 to 38%. As a result, excellent ductility and a low cycle fatigue life can be achieved.

Note that the content of the unavoidable impurity may be 0%. In the case where the content of the unavoidable impurity exceeds 0%, the composition ratio of the unavoidable impurity is adjusted so that the total is 100% on the basis of the composition ratio of Co, Ni, Cr, and Mo.

Cr (chromium) is an essential constituent for achieving the corrosion resistance and has an effect to strengthening the matrix. In the case where the unavoidable impurity is 0%, the content of Cr in the present invention is favorably 12 to 28%, more favorably 14 to 27%, and still more favorably 18 to 22%. Excellent corrosion resistance is likely to be achieved when the unavoidable impurity is 12% or more, and it is difficult for the workability and toughness to rapidly decrease when the unavoidable impurity is 28% or less. As a result, more excellent corrosion resistance is achieved while achieving the workability and toughness.

Ti (titanium) has effects of strong deoxidizing, denitrifying, and desulfurizing, but inclusions increase in an alloy and a η phase (Ni₃Ti) is precipitated to reduce the toughness when too much Ti is contained. For this reason, the content of Ti in the present invention is desirably 1.0% or less as the unavoidable impurity.

Mn (manganese) has effects of deoxidizing, desulfurizing, and stabilizing the face-centered cubic lattice phase, but the corrosion resistance and the oxidation resistance are deteriorated when too much Mn is contained. For this reason, the content of Mn in the present invention is desirably 1.5% or less. The upper limit thereof as the unavoidable impurity is more desirably 1.0% or less.

Fe (iron) has the functions of stabilizing the face-centered cubic lattice phase and improving the workability, but the oxidation resistance is reduced when too much Fe is contained. For this reason, the content of Fe in the present invention is desirably 1.0% or less as the unavoidable impurity.

C (carbon) has the effects of forming a carbide with Cr, Mo, or the like to prevent coarsening of crystal grains as well as being dissolved in the matrix, the toughness is reduced and the corrosion resistance is deteriorated when too much C is contained. For this reason, the content of C in the present invention is desirably 0.1% or less.

Nb (niobium) has the effects of being dissolved in the matrix to strengthen this and increasing the work hardenability, but a σ phase and a δ phase (Ni₃Nb) are precipitated to reduce the toughness when the content of Nb exceeds 3.0%. The content of Nb in the present invention is desirably 3.0% or less. The upper limit thereof as the unavoidable impurity is more desirably 1.0% or less.

W (tungsten) has the effects of being dissolved in the matrix to strengthen this and significantly increasing the work hardenability, but a σ phase is precipitated to reduce the toughness when the content of W exceeds 5.0%. For this reason, the content of W in the present invention is desirably 5.0% or less. The upper limit thereof as the unavoidable impurity is more desirably 1.0% or less.

Al (aluminum) has the effects of deoxidizing and improving the oxidation resistance, but the corrosion resistance is deteriorated when too much Al is contained. For this reason, the content of Al in the present invention is desirably 0.5% or less.

Zr (zirconium) has the effect of increasing the grain boundary strength at high temperatures to improve the hot workability, but the workability is deteriorated conversely when too much Zr is contained. For this reason, the content of Zr in the present invention is desirably 0.1% or less.

B (boron) has the effect of improving the hot workability but the hot workability is reduced conversely and cracks easily occurs when too much B is contained. For this reason, the content of B in the present invention is desirably 0.01% or less.

(Cobalt-Chromium Alloy as-Processed Material)

A cobalt-chromium alloy as-processed material according to the present invention is obtained by causing the cobalt-chromium alloy material to be subjected to cold working into a predetermined shape.

In the present invention, an fcc deformation twin and an hcp phase (F phase) are introduced by a twin deformation and an induced transformation during cold working, and a band-like deformed zone tissue having high density is formed. As a result, very high strength is achieved.

In addition, in the present invention, cold working refines the crystal grains and makes it easier to achieve higher strength.

The predetermined shape is not particularly limited, but is favorably a tubular shape or a wire shape, for example. This allows for use in a medical or aerospace device having a tubular shape or a wire shape. The cross-sectional shape of the wire shape includes a circular cross section, an oval cross section, a flat cross section, and a recessed or projecting irregular cross section. The tubular shape is hollow inside and has a circumferential surface surrounded by a cobalt-chromium alloy.

(Cobalt-Chromium Alloy Member)

A cobalt-chromium alloy member according to the present invention is obtained by performing specific heat treatment at a crystallization temperature or more on the cobalt-chromium alloy as-processed material.

By performing the heat treatment according to the present invention, the fcc deformation twin or the hcp phase in the cobalt-chromium alloy as-processed material changes to the fcc phase. Due to the formation of the fcc phase, an fcc twin deformation or a deformation-induced transformation from fcc to hcp occurs again when the cobalt-chromium alloy member is deformed. The cobalt-chromium alloy member according to the present invention, in which such a deformation or transformation occurs, has excellent mechanical strength and excellent ductility.

In addition, by performing the heat treatment according to the present invention, the crystal grains are made uniform and the mechanical properties thereof are homogenized.

In the cobalt-chromium alloy member according to the present invention, the tensile strength is 800 to 1200 MPa, favorably 850 to 1200 MPa.

In the cobalt-chromium alloy member, the uniform elongation is 25 to 60%, favorably 50 to 60%.

In the cobalt-chromium alloy member, the breaking elongation is 30 to 80%, favorably 60 to 80%.

The tensile strength, the uniform elongation, and the breaking elongation are measured by, for example, a tensile test using Autograph manufactured by Shimadzu Corporation.

The cobalt-chromium alloy member having the physical properties described above has excellent mechanical strength and excellent ductility.

The temperature of the heat treatment according to the present invention exceeds the recrystallization temperature of the cobalt-chromium alloy material and is not more than 1100° C., and is favorably 900° C. or more and 1100° C. or less.

By setting the temperature to the recrystallization temperature or more, recrystallization is performed and the fcc phase is stabilized. By setting the temperature to 1100° C. or less, the coarsening of the crystal grain size is suppressed.

As a result, a cobalt-chromium alloy member having a tensile strength, a uniform elongation, and a breaking elongation in the range described above, high mechanical strength, and high ductility is obtained.

The time of the heat treatment according to the present invention is 1 minute or more and 60 minutes or less. By setting the time to 1 minute or more, recrystallization is sufficiently performed and the fcc phase is stabilized. By setting the time to 60 minutes or less, the coarsening of the crystal grain size is suppressed.

As a result, a cobalt-chromium alloy member having a tensile strength, a uniform elongation, and a breaking elongation in the range described above, high mechanical strength, and high ductility is obtained.

The cobalt-chromium alloy member according to the present invention may have a crystal structure consisting of a face-centered cubic lattice (fcc) or a crystal structure consisting of a face-centered cubic lattice (fcc) and a hexagonal lattice (hcp).

As a result, an fcc twin deformation and a deformation-induced transformation from fcc to hcp are likely to occur when the cobalt-chromium alloy member is deformed, and more excellent mechanical strength and more excellent ductility are achieved.

The average value of the crystal grain size of the cobalt-chromium alloy member according to the present invention is favorably 5 μm or more and 30 μm or less, more favorably 7 μm or more and 10 μm or less. As a result, high mechanical strength is likely to be achieved.

The average value of the crystal grain size is calculated by an area-fraction method using electron backscatter diffraction (EBSD). In detail, the average value of the crystal grain size can be calculated in accordance with JIS G0551 “Steels-Micrographic determination of the apparent grain size” and ASTM E112-13 “Standard Test Methods for Determining Average Grain Size”.

The cobalt-chromium alloy member according to the present invention may include a band-like deformed zone tissue. The belt-like deformed band tissue according to the present invention is an aggregate tissue of dislocation cells in which a large number of dislocations generated by cold working are densely packed and is a tissue in the vicinity of the fcc deformation twin and the hcp phase (F phase) introduced during cold working.

The cobalt-chromium alloy member according to the present invention has a low stacking fault energy and achieves high work hardenability by the movement of partial dislocations and the formation of fine plate-like fcc twins and hcp phases during deformation. Further, high work hardenability is exhibited because solute atoms such as Mo and Nb having large or approximate atomic radii are strongly attracted to dislocation cores or stacking faults of extended dislocations to segregate, making it difficult for cross slip to occur, as compared with Co, Ni, and Cr whose atomic radius is 1.25 Å.

Further, since the work hardenability of the cobalt-chromium alloy member according to the present invention is exhibited not only at temperatures around the body temperature but also at high temperatures, it has characteristics of high high-temperature strength properties. Therefore, the use of the cobalt-chromium alloy member is not limited to medical use and withstands use under severe conditions, such as aerospace use and steam turbine use.

(Method of Producing Cobalt-Chromium Alloy Member)

A method of producing the cobalt-chromium alloy member includes a step of preparing a cobalt-chromium alloy material; a step of homogenizing the prepared cobalt-chromium alloy material at 1100° C. to 1300° C.; a step of causing the homogenized cobalt-chromium alloy material to be subjected to cold plastic working into a tubular shape or a wire shape to obtain a cobalt-chromium alloy as-processed material; and a step of performing heat treatment for 1 minute or more and 60 minutes or less at a temperature exceeding a recrystallization temperature of the cobalt-chromium alloy material and not more than 1100° C. on the cobalt-chromium alloy as-processed material that has been subjected to cold plastic working.

As a result, a cobalt-chromium alloy member having high mechanical strength and high ductility is obtained.

In the step of preparing a cobalt-chromium alloy material, the cobalt-chromium alloy material described above is used.

In the step of causing the homogenized cobalt-chromium alloy material to be subjected to cold plastic working, the cobalt-chromium alloy as-processed material that has been subjected to cold working into a tubular shape or a wire shape is obtained.

In the step of performing heat treatment on the cobalt-chromium alloy as-processed material, the cobalt-chromium alloy member is obtained.

In the homogenizing, by performing heat treatment on the cobalt-chromium alloy material at 1100° C. to 1300° C., each composition is uniformly dispersed. As a result, the uniformity of the mechanical properties is achieved in the cold working in the subsequent step.

By setting the homogenizing temperature to 1100° C. or more, it is possible to efficiently homogenize the material. By setting the homogenizing temperature to 1300° C. or less, it is possible to prevent the excessive coarsening of crystal grains and significant oxidation of the material surface. Other homogenizing conditions can be appropriately set within the range that does not impair the physical properties of the cobalt-chromium alloy member to be obtained.

The cobalt-chromium alloy material to be homogenized only needs to be a cobalt-chromium alloy material having the specific composition described above, and may be, for example, an alloy ingot prepared by high-frequency melting.

Further, the homogenized cobalt-chromium alloy material may be subjected to hot working into a shape that is easy to apply cold work, such as a round bar.

Further, in the method of producing a cobalt-chromium alloy member according to the present invention, after performing heat treatment at a temperature of the recrystallization temperature or more and 1100° C. or less on a cobalt-chromium alloy as-processed material obtained by causing a cobalt-chromium alloy material to be subjected to cold working into a plate material for a stent, aging treatment at a temperature of 200° C. or more and the recrystallization temperature or less may be performed. As a result, higher strength properties are achieved by so-called static strain aging, in which solute atoms such as Mo are attracted to dislocation cores or stacking faults of extended dislocations to fix the dislocations.

### Example

In order to achieve the above-mentioned object, a cobalt-chromium alloy material having a composition of, in terms of mass %, 23 to 32% of Ni, 37 to 48% of Co, and 8 to 12% of Mo, a remainder thereof containing Cr and an unavoidable impurity, the composition satisfying a relationship of

20≤[Cr %]+[Mo %]+[unavoidable impurity %]≤40,

was adopted.

An alloy ingot having the composition of this cobalt-chromium alloy material was prepared by high-frequency melting, and hot forging and homogenizing were performed thereon at 1100° C. to 1300° C. to produce a round bar having a diameter of 8 mm and a length of 270 mm by hot rolling and cutting. This round bar corresponds to a cobalt-chromium alloy material.

Next, this cobalt-chromium alloy material was subjected to cold working to obtain a tube material having a diameter of 1.6 mm, a thickness of 0.1 mm, and a length of 1 m. This tube material corresponds to a cobalt-chromium alloy as-processed material. Further, predetermined heat treatment was performed on this tube material to impart ductility thereon, thereby obtaining a cobalt-chromium alloy member as a tube material.

Further, the cobalt-chromium alloy material was subjected to cold working to obtain a wire material having a diameter of 0.5 mm and a length of 1 m. This wire material corresponds to a cobalt-chromium alloy as-processed material. Further, predetermined heat treatment was performed on this wire material to impart ductility thereon, thereby obtaining a cobalt-chromium alloy member as a wire material.

The composition of the cobalt-chromium alloy material used in this Example is shown in Table 1. The unit is mass %.

TABLE 1

| | | | | | | | Unavoidable |
|---|---|---|---|---|---|---|---|
| | Co | Cr | Mo | Ni | W | Fe | impurity |
| Example 1 | 38 | 20 | 10 | 32 | 0 | 0 | 0 |
| Example 2 | 41 | 20 | 10 | 29 | 0 | 0 | 0 |
| Example 3 | 44 | 20 | 10 | 26 | 0 | 0 | 0 |
| Example 4 | 47 | 20 | 10 | 23 | 0 | 0 | 0 |
| Comparative | 35 | 20 | 10 | 35 | 0 | 0 | 0 |
| Example 1 | | | | | | | |
| Comparative | 50 | 20 | 10 | 20 | 0 | 0 | 0 |
| Example 2 | | | | | | | |
| Comparative | 55 | 20 | 0 | 10 | 15 | 0 | 0 |
| Example 3 | | | | | | | |
| Comparative | 18 | 0 | 2 | 12 | 0 | 68 | 0 |
| Example 4 | | | | | | | |

In Examples 1 to 4, the contents of Cr and Mo were respectively made constant, i.e., 20 mass % and 10 mass %, and the content of Co was changed with respect to the content of Ni. The content of Ni was changed in the range of 23 to 32 mass %.

In Comparative Examples 1 to 4, commercially available Co-20Cr-10Mo-35Ni alloy (hereinafter, referred to simply as an “MP35N alloy”), Co-20Cr-10Mo-20Ni alloy, Co-20Cr-15W-10Ni alloy (hereinafter, referred to simply as an “L605 alloy”), and SUS316L (manufactured by Hayes) were used as comparative materials, respectively.

A low cycle fatigue test at a strain amplitude of 0.01 was performed on the cobalt-chromium alloy materials having the compositions according to the Examples 1 to 4 and the alloys having the compositions according to the Comparative Examples 1 to 4, which have been subjected to hot working into a rod shape and then heat-treated at 1200° C. for 1 minute.

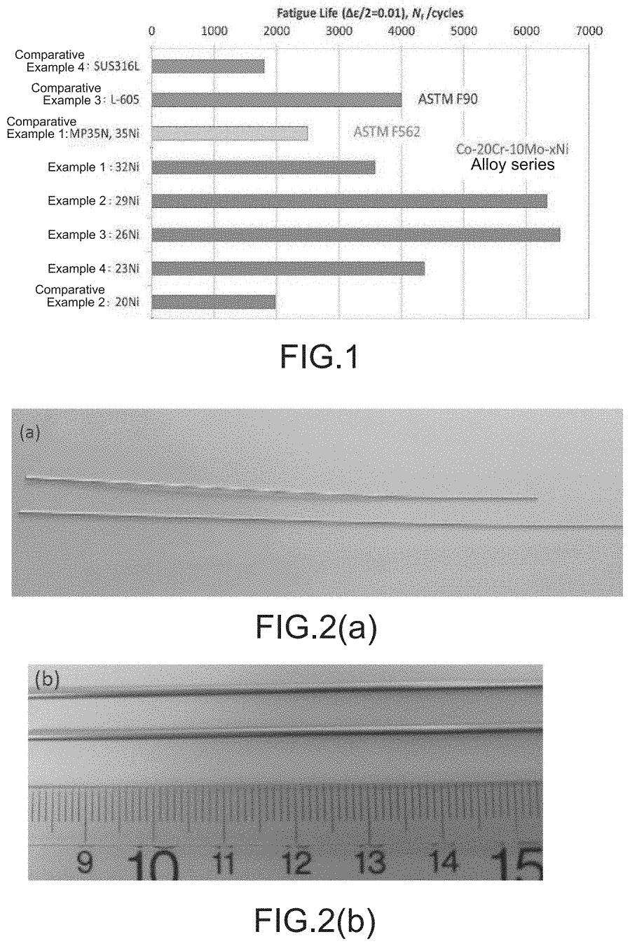

The test results are shown in FIG. 1. In each of the Examples 1 to 4, the fatigue life was favorable, i.e., 3000 cycles or more. In particular, each of the cobalt-chromium alloy materials having 23 mass % of Ni (Example 4), 26 mass % of Ni (Example 3), and 29 mass % of Ni (Example 2) had an improved low cycle fatigue life as compared with the ready-made products according to the Comparative Examples 1 to 4.

Further, a tensile strength test at a stain rate of 2.5×10⁻⁴ s⁻¹ was performed using a tensile tester manufactured by A&D Company, Limited on the cobalt-chromium alloy materials having the compositions according to the Examples 1 to 4 and the alloys having the compositions according to the Comparative Examples 1 to 4, which have been subjected to hot working into a rod shape and then heat-treated at 1200° C. for 1 minute, and the results thereof were shown in Table 2. In the cobalt-chromium alloy materials according to the Examples 1 to 4, tensile strengths of 848 to 886 MPa were shown and high tensile strengths peculiar to the cobalt-chromium alloys equivalent to that of the MP35N alloy (Comparative Example 1) were shown.

TABLE 2

| Cobalt-chromium | Tensile strength | Breaking elongation |
|---|---|---|
| alloy material | [MPa] | [%] |
| Example 1 | 850 | 71 |
| Example 2 | 848 | 73 |
| Example 3 | 880 | 77 |
| Example 4 | 886 | 61 |
| Comparative | 823 | 20 |
| Example 1 | | |
| Comparative | 845 | 20 |
| Example 2 | | |
| Comparative | 1034 | 40 |
| Example 3 | | |
| Comparative | 515 | 60 |
| Example 4 | | |

FIG. 2 shows appearance photographs of a tube as a cobalt-chromium alloy as-processed material (top) prepared by causing a Co-20Cr-10Mo-26Ni alloy, which has the most excellent fatigue life among cobalt-chromium alloy materials, to be subjected to cold working and a cobalt-chromium alloy member (bottom) on which heat treatment has been performed for 5 minutes at 1050° C., Part (a) being a whole photograph, Part (b) being an enlarged photograph of the main portion. The size thereof is an outer diameter of 1.6 mm, a thickness of 0.1 mm, and a length of 980 to 1280 mm, and it has favorable surface properties.

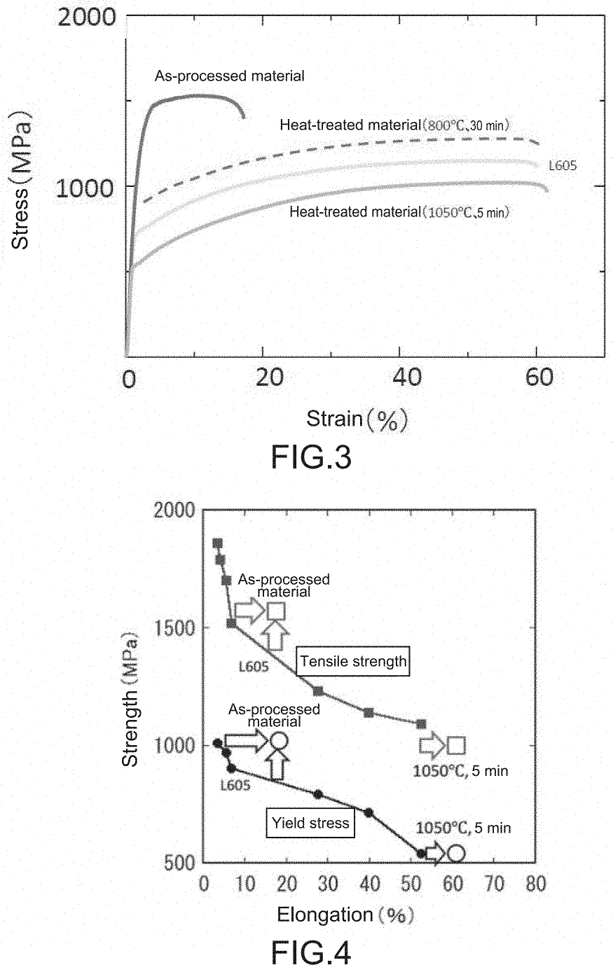

FIG. 3 is a diagram showing the results of measuring the tensile strength of a cobalt-chromium alloy as-processed material (hereinafter, referred to simply as an “as-processed material”) that has been subjected to cold working, which is a tube material of the prepared Co-20Cr-10Mo-26Ni alloy, a cobalt-chromium alloy member (hereinafter, referred simply as a “heat-treated material”) obtained by performing heat treatment for 5 minutes at 1050° C. on the as-processed material, and an L605 alloy tube that is a comparative material. The horizontal axis indicates the strain [%] and the vertical axis indicates the stress [MPa]. The test was performed using UTM-III-500 manufactured by TOYO BALDWIN CO. Ltd. at a test speed of 5 mm/min and a gauge length: 20 mm.

Further, Table 3 shows the 0.2% proof stress [MPa], the tensile strength [MPa], the uniform elongation [%], and the breaking elongation [%] obtained from FIG. 3. The heat-treated material obtained by performing heat treatment at 1050° C. for 5 minutes on the as-processed material after cold working had values of a uniform elongation and a breaking elongation larger than those of the L605 alloy tube. The broken line in FIG. 3 shows a stress-strain diagram drawn with reference to the tensile strength obtained from the hardness measurement for the heat-treated material obtained by performing heat treatment at 800° C. for 30 minutes on the as-processed material.

TABLE 3

| | 0.2% | | | |
|---|---|---|---|---|
| | proof | Tensile | Unform | Breaking |
| | stress | strength | elongation | elongation |
| | (MPa) | (MPa) | (%) | (%) |
| As-processed material | 1115 | 1530 | 10.4 | 17.3 |
| Heat-treated material | 523 | 1021 | 55.8 | 61.6 |
| (1050° C., 5 min) | | | | |
| Comparative material | 694 | 1149 | 52.4 | 60.2 |
| L605 tube | | | | |
| (Manufactured by | | | | |
| Minitubes) | | | | |

FIG. 4 is a diagram comparing values of a yield stress, a tensile strength, and an elongation of a tube material (as-processed material) of a Co-20Cr-10Mo-26Ni alloy and a heat-treated material obtained by performing heat treatment at 1050° C. for 5 minutes with literature values of the L605 alloy (see Non-Patent Literature 2). The vertical axis indicates the strength [MPa] and the horizontal axis indicates the elongation [%]. As compared with the literature values, the yield stress of the tube material (as-processed material) according to the present invention is higher than that of the L605 alloy tube exhibiting a comparable elongation. Further, it exhibits an elongation larger than that of L605 exhibiting a comparable yield stress. Further, the heat-treated material obtained by performing heat treatment at 1050° C. for 5 minutes exhibits an elongation larger than that of the L605 alloy exhibiting a comparable yield stress.

The cobalt-chromium alloy as-processed material (tube material) according to an Example of the present invention exhibits a tensile strength higher than that of the L605 alloy exhibiting a comparable elongation. Further, the heat-treated material having a tubular shape on which heat treatment at 1050° C. for 5 minutes has been performed, which is a cobalt-chromium alloy member according to the present invention, exhibits an elongation larger than that of the L605 alloy having a comparable tensile strength (FIG. 4).

Regarding the tube material (as-processed material) obtained by causing the cobalt-chromium alloy material according to an Example of the present invention to be subjected to cold working, when the yield stress and the tensile strength of the cobalt-chromium alloy as-processed material according to the present invention are compared with the literature values of those of the L605 alloy tube, the yield stress of the cobalt-chromium alloy as-processed material (tube material) according to the present invention is higher than that of the L605 alloy tube exhibiting a comparable elongation. Further, the cobalt-chromium alloy as-processed material according to the present invention exhibits an elongation larger than that of L605 exhibiting a comparable yield stress. Further, the heat-treated material obtained by heat treatment at 1050° C. for 5 minutes, which is a cobalt-chromium alloy member according to the present invention, exhibits an elongation larger than that of the L605 alloy exhibiting a comparable yield stress.

Table 4 shows the Micro-Vickers hardness [HV] and the tensile strength [MPa] of the materials obtained by performing heat treatment on the cobalt-chromium alloy as-processed material at 1000° C. for 60 minutes, 1000° C. for 30 minutes, 800° C. for 30 minutes, 600° C. for 30 minutes, and 400° C. for 30 minutes.

TABLE 4

| | Hardness | Tensile strength |
|---|---|---|
| | (Hv) | (MPa) |
| As-processed material | 490.6 | 1602 |
| 1000° C., 60 minutes | 284.2 | 928.4 |
| 1000° C., 30 minutes | 299.4 | 978 |
| 800° C., 30 minutes | 360.7 | 1178.3 |
| 600° C., 30 minutes | 581.2 | 1898.6 |
| 400° C., 30 minutes | 502.2 | 1640.5 |

The hardness measurement was performed with a weight of 50 g and the load time of 15 seconds. The tensile strength was calculated using the following conversion formula (see Non-Patent Literature 1).

Tensile strength=Hardness×9.8/3

In the Co-20Cr-10Mo-26Ni alloy, when performing heat treatment after cold working, the hardness had a value lower than that of the as-processed material and the tensile strength was in the range of 800 to 1200 MPa at 800° C. or more, which is the crystallization temperature or more. Meanwhile, in the case of heat treatment of 600° C. or less, which is lower than the crystallization temperature, the hardness had substantially the same value as or a value higher than that of the as-processed material and the tensile strength exceeded 1200 MPa.

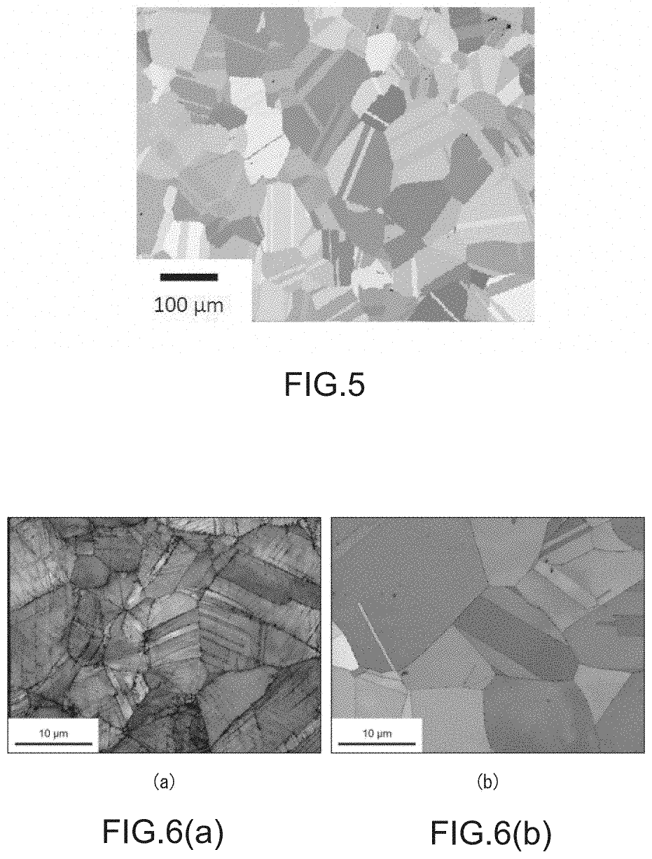

FIG. 5 is a crystal orientation map obtained by EBSD showing crystal grains of the Co-20Cr-10Mo-26Ni alloy before cold working. The average value of the crystal grain size was approximately 30 m.

Note that the average value of the crystal grain size was measured in accordance with ASTM E112-13 “Standard Test Methods for Determining Average Grain Size”.

FIG. 6 shows inverse pole maps obtained by an electron backscatter diffraction (EBSD) method of the as-processed material prepared by causing the cobalt-chromium alloy material to be subjected to cold working into a tubular shape and the heat-treated material thereof.

Part (a) of FIG. 6 is an inverse pole map obtained by an electron backscatter diffraction (EBSD) method showing the tissue of the as-processed material having a tubular shape of the Co-20Cr-10Mo-26Ni alloy (Example 3) after adjusting the surface state. The crystal grains were fine, i.e., the average value of the crystal grain size was approximately 10 μm or less, and a band-like deformed zone tissue having high density was observed. These belt-like tissues were hcp phases (ε phases) or deformation twins introduced by plastic working. Part (b) of FIG. 6 is an inverse pole map image of the material obtained by performing heat treatment at 1050° C. for 5 minutes on the as-processed material. The average value of the crystal grain size was approximately 20 μm, which is larger than that of the as-processed material, and the number of deformed bands was reduced. That is, an fcc phase is formed in this heat-treated material, and an hcp phase (F phase) or a deformation twin is introduced again when this heat-treated material is deformed, thereby increasing the number of belt-like deformed band tissues. In the cobalt-chromium alloy member according to the present invention, which changes in this way, high strength and high ductility are achieved.

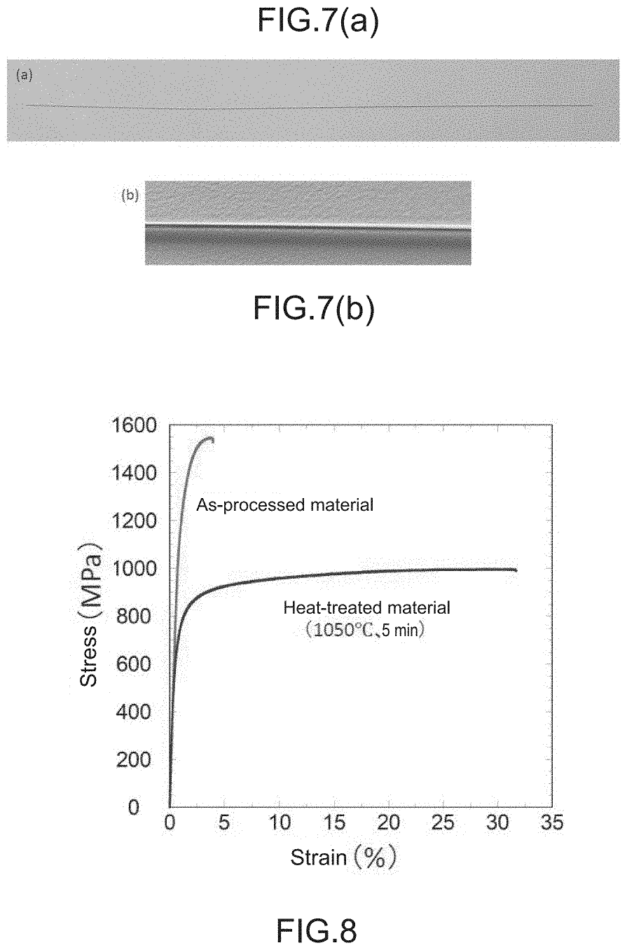

FIG. 7 shows appearance photographs of the cobalt-chromium alloy as-processed material having a wire shape, which is prepared by cold working, Part (a) being a whole photograph, Part (b) being an enlarged photograph of the main portion. It has a diameter of 0.5 mm, a length of 1000 mm, and a favorable appearance.

FIG. 8 is a diagram showing the results of measuring the tensile strength of those obtained by performing heat treatment 1050° C. for 5 minutes on the prepared cobalt chromium alloy as-processed material having a wire shape of the Co-20Cr-10Mo-26Ni alloy. The horizontal axis indicates the strain [%] and the vertical axis indicates the stress [MPa]. The tensile test was performed at a test speed of 1.2 mm/s and a gauge length of 110 mm using Autograph manufactured by Shimadzu Corporation.

Table 5 compares the tensile strength and the breaking elongation of the wire as a cobalt-chromium alloy member according to an Example of the present invention with those of SUS316L, the L605 alloy, and the MP35N alloy.

TABLE 5

| | | Tensile | Breaking |
|---|---|---|---|
| | | strength | elongation |
| | | (MPa) | (%) |
| Present | As-processed | 1542 | 4 |
| invention | material | | |
| | 1050° C. | 996 | 32 |
| | 5 min | | |
| | Heat-treated | | |
| | material | | |
| Comparative | Wire (0%) | 1138 | 50 |
| Example | Wire | 1655 | 9 |
| L605 | (20%) | | |
| | Wire | 2000 | 6 |
| | (37%) | | |
| | Wire | 2241 | 3.6 |
| | (50%) | | |
| Comparative | Wire | 1048 | 50 |
| Example | CW 0% | | |
| MP35N | Wire | 2378 | 3.4 |
| | CW 90% | | |

### Comparative Example SUS316L: A Tensile Strength of 480 MPa and a Breaking Elongation of 40%

The numerical values (%) of “Comparative Example L605” and “Comparative Example MP35N” in the table indicate the rate of cold working.

The wire as a cobalt-chromium alloy member according to an Example of the present invention exhibited a strength exceeding that of SUS316L that is most widely used as a guide wire and exhibited a tensile strength and a breaking elongation comparable to those of the wires of the L605 alloy and MP35N (FIG. 8, Table 5).

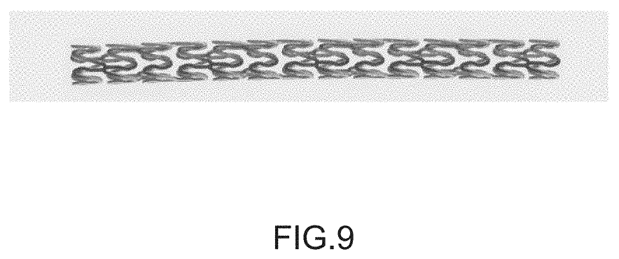

FIG. 9 shows a stent cut out from a tube that includes a cobalt-chromium alloy member of a Co-20Cr-10Mo-26Ni alloy as a cobalt-chromium alloy member according to an Example of the present invention by a laser processing device. It has a favorable appearance and favorable laser processability.

### INDUSTRIAL APPLICABILITY

As described above in detail, a cobalt-chromium alloy member having high strength and high ductility is obtained by preparing a cobalt-chromium alloy material having an alloy composition according to the present invention into a predetermined shape such as a tube and a wire by cold working and then performing heat treatment exceeding the recrystallization temperature of the cobalt alloy material thereon. Such a cobalt-chromium alloy member is suitable for use in a medical device and an aerospace device because it uses a cobalt-chromium alloy member having a long fatigue life.

Examples of the medical device include indwelling medical devices such as a stent, a catheter, a fastening cable, a guide rod, an orthopedic cable, a heart valve, and an implant. As another medical device, it can be used as a bone drill bit and a gallstone removal wire.

Examples of the aerospace device include a corrosion resistant shield cable, a high-performance wire, and a high-performance cable. Examples of the industrial device include a precision wire, which is used as a brush seal of a steam turbine.

1. A cobalt-chromium alloy member, which is obtained by performing heat treatment for 1 minute or more and 60 minutes or less at a temperature exceeding a recrystallization temperature of a cobalt-chromium alloy material and not more than 1100° C. on a cobalt-chromium alloy as-processed material obtained by causing the cobalt-chromium alloy material to be subjected to cold plastic working into a predetermined shape, the cobalt-chromium alloy material having a composition of,
in terms of mass %, 23 to 32% of Ni, 37 to 48% of Co, and 8 to 12% of Mo, a remainder thereof containing Cr and an unavoidable impurity, the composition satisfying a relationship of
20≤[Cr %]+[Mo %]+[unavoidable impurity %]≤40,
the cobalt-chromium alloy member having a tensile strength of 800 to 1200 MPa, a uniform elongation of 25 to 60%, and a breaking elongation of 30 to 80%.

2. The cobalt-chromium alloy member according to claim 1, which is obtained by performing heat treatment for 1 minute or more and 60 minutes or less at a temperature of 900° C. or more and 1100° C. or less on a cobalt-chromium alloy as-processed material obtained by causing the cobalt-chromium alloy material to be subjected to cold plastic working into a predetermined shape, the cobalt-chromium alloy material having a composition of,
in terms of mass %, 25 to 29% of Ni, 37 to 48% of Co, and 9 to 11% of Mo, a remainder thereof containing Cr and an unavoidable impurity, the composition satisfying a relationship of
23≤[Cr %]+[Mo %]+[unavoidable impurity %]≤38,
the cobalt-chromium alloy member having a tensile strength of 850 to 1200 MPa, a uniform elongation of 50 to 60%, and a breaking elongation of 60 to 80%.

3. The cobalt-chromium alloy member according to claim 1, wherein
the unavoidable impurity contains, in terms of mass %, 1.0% or less of Ti, 1.0% or less of Mn, 1.0% or less of Fe, 1.0% or less of Nb, 1.0% or less of W, 0.5% or less of Al, 0.1% or less of Zr, 0.01% or less of B, and 0.1% or less of C as contents of Ti, Mn, Fe, Nb, W, Al, Zr, B, and C.

4. The cobalt-chromium alloy member according to claim 1, which has a crystal structure consisting of a face-centered cubic lattice (fcc) or a crystal structure consisting of a face-centered cubic lattice (fcc) and a hexagonal lattice (hcp), an average crystal grain size of 5 to 30 μm, and a band-like deformed zone tissue.

5. A medical device or an aerospace device, which uses the cobalt-chromium alloy member according to claim 1.

6. The medical device according to claim 5, which includes a stent, a tube, a wire, or an implant.

7. A method of producing a cobalt-chromium alloy member, characterized by comprising:
a cobalt-chromium alloy material having a composition of,
in terms of mass %, 23 to 32% of Ni, 37 to 48% of Co, and 8 to 12% of Mo, a remainder thereof containing Cr and an unavoidable impurity, the composition satisfying a relationship of
20≤[Cr %]+[Mo %]+[unavoidable impurity %]≤40;
homogenizing the prepared cobalt-chromium alloy material at 1100° C. to 1300° C.;
causing the homogenized cobalt-chromium alloy material to be subjected to cold plastic working into a tubular shape or a wire shape to obtain a cobalt-chromium alloy as-processed material; and
performing heat treatment for 1 minute or more and 60 minutes or less at a temperature exceeding a recrystallization temperature of the cobalt-chromium alloy material and not more than 1100° C. on the cobalt-chromium alloy as-processed material that has been subjected to cold plastic working.

8. The cobalt-chromium alloy member according to claim 2, wherein
the unavoidable impurity contains, in terms of mass %, 1.0% or less of Ti, 1.0% or less of Mn, 1.0% or less of Fe, 1.0% or less of Nb, 1.0% or less of W, 0.5% or less of Al, 0.1% or less of Zr, 0.01% or less of B, and 0.1% or less of C as contents of Ti, Mn, Fe, Nb, W, Al, Zr, B, and C.
